(19) United States
(12) Patent Application Publication
(10) Pub. No.: US 2019/0369015 A1
(43) Pub. Date: Dec. 5, 2019

(54) IDENTIFICATION OF MICROORGANISMS USING DISPOSABLE DUAL REFLECTION SUBSTRATE FOR MEASURING INFRARED SPECTRA OF SAID MICROORGANISMS

(71) Applicant: IRID CORP., Westmount (CA)

(72) Inventors: Ashraf A. ISMAIL, Westmount (CA); Jacqueline SEDMAN, Kirkland (CA)

(21) Appl. No.: 16/429,287

(22) Filed: Jun. 3, 2019

Related U.S. Application Data
(60) Provisional application No. 62/679,241, filed on Jun. 1, 2018.

(51) Int. Cl. G01N 21/3577; G01N 33/487

(52) U.S. Cl. CPC G01N 21/3577; G01N 33/48735

(57) ABSTRACT

The present disclosure presents methods and systems for the spectral identification of microorganisms using disposable or recyclable transflection and internal reflection infrared substrates. A background spectrum to measure a water vapor level of an ambient atmosphere in the absence of a sample is acquired. The sample containing the microorganism is brought into contact with a disposable infrared internal reflection substrate. The sample has intact microbial cells. Spectral data is acquired from the sample using internal reflection infrared spectroscopy or transflection infrared spectroscopy no more than a predetermined time after having acquired the background spectrum. The background spectrum and the spectral data are combined thereby producing modified spectral data. The microorganism is characterized using the modified spectral data.

### CROSS-REFERENCE TO RELATED APPLICATIONS

The present application claims priority to U.S. Provisional Patent Application bearing Ser. No. 62/679,241 filed on Jun. 1, 2018, the contents of which are hereby incorporated by reference.

### TECHNICAL FIELD

The present disclosure relates generally to analyzing microorganisms using spectral data obtained from infrared spectroscopy, and particularly to microbial differentiation and identification using infrared spectroscopy from a disposable device cable of accommodating single or multiple microorganisms.

### BACKGROUND OF THE ART

The use of infrared spectroscopy for microbial differentiation and identification dates back to 1954. The feasibility of such application of infrared spectroscopy was substantially enhanced by the advent of Fourier transform infrared (FTIR) spectroscopy and has been extensively investigated by numerous research groups over the past three decades. Taken together, this body of research indicates that the infrared spectra of pure microbial colonies serve as whole-organism fingerprints that are specific down to the subspecies level of taxonomic classification. However, the reliability of infrared spectroscopy as a means of microbial identification is dependent upon all the conditions employed in the identification procedure, beginning with growth of the microorganisms on culture media to obtain pure colonies and followed by sample preparation for infrared spectroscopic measurement, which entails the deposition of microbial cells, taken from one or more pure colonies, as a thin film on a suitable substrate.

FTIR spectra of microorganisms are commonly acquired in the transmission mode, although various other techniques such as attenuated total reflectance (ATR) and transflection spectroscopy have also been employed. For spectra acquired in the transmission mode, spectral reproducibility depends mainly on the uniformity of the sample (sample homogeneity, particle size) and sample thickness (or path length). Sample non-uniformity leads to baseline variations owing to the scattering, diffraction, and refraction that occur as the infrared (IR) beam passes through the sample, whereas variations in sample thickness result in variations in band intensity, although consistency in relative peak intensities is maintained. This limitation has been addressed with the use of infrared imaging microscopy.

Another limitation is the need for a large number of microbial cells to acquire a representative infrared spectrum of adequate quality. To accomplish this, the bacteria typically must be cultured for an extended period of time (16-24 hours). This limitation has been addressed with the use of infrared imaging microscopy.

The capital cost of infrared microscopy instrumentation is very high due to the need of using an infrared reflective microscope and a liquid-nitrogen cooled detector. This make the technology less accessible for routine use by medium and small microbiology laboratories.

There is therefore a need for improved methods for identifying microorganisms using spectral data in a more sensitive and cost effective manner.

### SUMMARY

The present disclosure presents methods and systems for the spectral identification of microorganisms using a disposable infrared substrate that may be employed in acquiring infrared spectra by attenuated total reflectance infrared (ATR-IR) spectroscopy or by transflection infrared (TFL-IR) spectroscopy.

The disposable substrate may be provided to acquire infrared spectra of microorganisms by attenuated total reflectance infrared (ATR-IR) spectroscopy. The disposable substrate may be provided to acquire infrared spectra of microorganisms by transflection infrared (TFL-IR) spectroscopy.

In some embodiments, a low cost disposable substrate is used to acquire spectral of a limited amount of microbial cells without the need for any reagents.

In some embodiments, a low cost disposable substrate is used to acquire spectral of a limited amount of microbial cells without the need for any reagents in combination with a low cost infrared imaging detector operating at ambient (or sub-ambient temperatures without the need for liquid nitrogen).

In some embodiments, a low cost disposable substrate is used to acquire spectral of a limited amount of microbial cells without the need for any reagents in combination with a low cost infrared imaging detector operating at ambient (or sub-ambient temperatures without the need for liquid nitrogen) or use of an infrared microscope.

In accordance with a broad aspect, there is provided a method for spectral identification of a microorganism. The method comprises acquiring a background spectrum to measure a water vapor level of an ambient atmosphere in the absence of a sample, bringing the sample containing the microorganism into contact with a disposable infrared substrate, the sample having intact microbial cells, acquiring spectral data from the sample, using at least one of internal reflection infrared spectroscopy or transflection infrared spectroscopy, no more than a predetermined time after having acquired the background spectrum, combining the background spectrum and the spectral data, thereby producing modified spectral data, and characterizing the microorganism using the modified spectral data.

In some embodiments, the disposable infrared substrate is capable prorogating infrared light therethrough which results in an internal reflection process. Thus, in some embodiments, disposable infrared substrate is a disposable infrared internal substrate.

In accordance with another broad aspect, there is provided a method for spectral identification of a microorganism. The method comprising: acquiring a background spectrum to measure a water vapor level of an ambient atmosphere in the absence of a sample, bringing the sample containing the microorganism into contact with a disposable infrared internal reflection substrate coated with an infrared reflective coating, the sample having intact microbial cells, acquiring spectral data from the sample using transflection infrared spectroscopy no more than a predetermined time after having acquired the background spectrum, combining the background spectrum and the spectral data, thereby producing modified spectral data, and characterizing the microorganism using the modified spectral data.

In accordance with another broad aspect, there is provided a method for spectral identification of a microorganism. The method comprising acquiring a background spectrum to measure a water vapor level of an ambient atmosphere in the absence of a sample, bringing the sample containing the microorganism into contact with a disposable infrared internal reflection substrate, the sample having intact microbial cells, acquiring spectral data from the sample using internal reflection infrared spectroscopy no more than a predetermined time after having acquired the background spectrum, combining the background spectrum and the spectral data, thereby producing modified spectral data, and characterizing the microorganism using the modified spectral data.

In some embodiments, substrate materials can be composed of any one of: silicon, germanium, zinc selenide, amorphous materials transmitting infrared (AMTIR), thallium bromoidodide (KRS 5), chalcogenides (e.g., chalcogenide glass), halide salts, synthetic diamond film or wafers and any other suitable mid-infrared transmission materials.

In some embodiments, the disposable infrared substrate is uncoated. In some embodiments, disposable infrared substrate is coated. The disposable infrared substrate may be coated with an infrared reflective thin coating. The infrared reflective thin coating may be a thin layer of an infrared reflective material such as indium-tin-oxide, or a metal (e.g., gold, aluminum, silver or the like). The thin coating may be vapor deposited or chemically deposited on the disposable infrared substrate.

In some embodiments, at least one thin polymer material is added to infrared internal reflection substrate or the infrared reflective thin coating for attached biomolecules to concentrate the microorganisms near a surface of the internal reflection substrate.

In some embodiments, the disposable infrared internal reflection substrate comprises one or more microfluidic device for allowing simultaneous separation of the microorganism from the sample. The one or more microfluidic devices may be single channel or multiple channel.

In some embodiments, the spectral data is acquired from the sample prior to or after having added a MALDI-TOF chemical matrix thereto.

In some embodiments, the sample has a limited free water content and an intact associated and bound water content.

In some embodiments, the microorganism in the sample has a water activity less than 0.999%.

In some embodiments, the method further comprises applying a vacuum to the sample on the disposable infrared substrate prior to acquiring the spectral data from the sample using at least one of ATR-IR or TFL-IR infrared spectroscopy.

In some embodiments, the method further comprises recording the infrared spectra employing a single infrared detector. In some embodiments, the method further comprises recording the infrared spectra employing a plurality of infrared detectors. The infrared detector(s) may operate at ambient or sub ambient temperatures.

In some embodiments, the method further comprises recording the infrared spectra employing an infrared array detector operating at room temperature (or sub ambient temperatures).

In some embodiments, the method further comprises recording the infrared spectra employing a Michelson interferometer to generate an infrared modulated infrared light.

In some embodiments, the method further comprises recording the infrared spectra employing a Fabry-Pérot interferometer (FPI) to generate an infrared modulated infrared light.

In some embodiments, the method further comprises recording the infrared spectra employing a linear variable array detector.

In some embodiments, the method further comprises recording the infrared spectra employing a quantum cascade laser to generate discreet infrared wavelengths.

In some embodiments, the method further comprises recording the infrared spectra employing an x-y or an x-y-z stage to acquire spectra from multiple samples deposited on the disposable infrared substrate.

In some embodiments, the method further comprises recording the infrared spectra employing a computer controlled x-y or an x-y-z stage to acquire spectra from multiple samples deposited on the disposable infrared substrate.

In some embodiments, the method further comprises recording the infrared spectra from a microfluidic device comprised in part of the disposable infrared substrate.

In some embodiments, the method further comprises recording the infrared spectra from a microfluidic device to isolate microorganism from a fluid specimen comprised in part of the disposable infrared substrate.

In some embodiments, the method further comprises recording the infrared spectra from a microfluidic device to isolate microorganisms from a fluid specimen comprised in part of the disposable infrared substrate. The fluid specimen may be from blood, urine, sputum or any other bodily fluids.

In some embodiments, the method further comprises recording the infrared spectra from a microfluidic device to isolate microorganisms from a fluid specimen comprised in part of the disposable infrared substrate. The fluid specimen may be from blood, urine, sputum or any other bodily fluids. The infrared spectra of the isolated microorganisms may be measured directly from the microfluidic device by either of both infrared transflection or infrared total internal reflection spectroscopy.

In accordance with another broad aspect, there is provided a system for spectral identification of a microorganism. The system comprises a processing unit and a non-transitory computer-readable memory having stored thereon program instructions. The program instructions are executable for acquiring a background spectrum to measure a water vapor level of an ambient atmosphere in the absence of a sample, acquiring spectral data from the sample, using at least one of ATR-IR or TFL-IR infrared spectroscopy, no more than a predetermined time after having acquired the background spectrum, the sample having been brought into contact with a disposable infrared reflective substrate and having intact microbial cells, combining the background spectrum and the spectral data, thereby producing modified spectral data, and characterizing the microorganism using the modified spectral data.

The above disposable devices, methods and/or systems of applying different spectral acquisition techniques may be extended to diagnosis of clinical specimens not limited to tissue or bodily fluids (urine, blood, sputum or the like).

The above disposable devices, methods and/or systems of applying different spectral acquisition techniques may be extended to diagnosis of clinical specimens not limited to microorganisms or bodily fluids (urine, blood, sputum or the like) as part a microfluidics devices (both single channel and multichannel configuration).

### BRIEF DESCRIPTION OF THE DRAWINGS

Further features and advantages of the present invention will become apparent from the following detailed description, taken in combination with the appended drawings, in which:

FIG. 1A is a diagram of an example setup for reflection infrared spectroscopy of a microorganism with a disposable substrate;

FIG. 1B is a schematic of the setup of FIG. 1A for attenuated total reflection infrared (ATR-IR) spectroscopy with the disposable substrate;

FIG. 2A is a flowchart of an example embodiment for a method of identifying microorganisms using reflection infrared (IR) spectroscopy with a disposable substrate:

FIG. 2B is a baseline spectrum record by the disposable ATR-IR substrate showing the signal-to-noise ratio (SNR) recorded using a portable Fourier transform infrared (FTIR) spectrometer in a spectral region nominally employed in the differentiation between microorganisms;

FIG. 3 illustrates an infrared spectrum of water deposited on a disposable ATR-IR substrate using a FTIR spectrometer;

FIG. 4 illustrates the water spectrum of FIG. 3 and a spectrum of a microorganism each deposited on a disposable ATR-IR substrate using the FTIR spectrometer;

FIG. 5 illustrates a comparison between the spectra of Listeria grayi recorded on a commercially available single-bounce diamond ATR-FTIR single-detector spectrometer (lower pane) and recorded using the disposable ATR-IR substrate coupled to an FTIR single-detector spectrometer;

FIG. 6 is a schematic of the setup of FIG. 1A for transflection infrared (TFL-IR) spectroscopy with the disposable substrate;

FIG. 7 illustrates a baseline spectrum record by a disposable TFL-IR substrate showing the signal-to-noise ratio (SNR) recorded using a FTIR spectrometer in a spectral region nominally employed in the differentiation between microorganisms;

FIG. 8 illustrates TFL-IR spectra of a S. aureus and S. epidermis deposited on a disposable substrate;

FIG. 9 illustrates spectrum recorded for a layer of thick emulsion deposited on a disposable IR substrate;

FIG. 10 illustrates a TFL-IR spectrum and an ATR-IR spectrum recorded on a disposable substrate of a thin ink layer;

FIG. 11A illustrates a chemical image generated by plot of the amide I band in the infrared spectral spectra of Listeria grayi recorded using a focal plane array detector FTIR spectrometer;

FIG. 11B illustrates a principal component plot demonstrating the discrimination between E. coli and Listeria grayi based on spectral differences acquired by FPA-FTIR infrared imaging microscopy;

FIG. 12 illustrates an example of the use of a dual purpose disposable transflection infrared substrate integrated in a multi-channel microfluidic device for analysis of biological samples, where the infrared spectra is recorded in a TFL-IR mode;

FIG. 13 illustrates example of the use of a dual purpose disposable attenuated total reflectance infrared substrate integrated into a multi-channel microfluidic device for analysis of biological samples, where the infrared spectra is recorded in an ATR-IR configuration;

FIG. 14 is an example system for spectral identification of microorganisms using reflection IR spectroscopy;

FIG. 15 is an example embodiment for a microorganism identification device;

FIG. 16 is an example embodiment of an application running on the microorganism identification device of FIG. 15;

FIG. 17A illustrates an example of use of a dual surface infrared substrate for recording spectra from a enterococci and staphylococci species directly from a nutrient agar; and

FIG. 17B illustrates an example of an infrared measurement for identifying microorganisms from a hydrophobic membrane filter.

It will be noted that throughout the appended drawings, like features are identified by like reference numerals.

### DETAILED DESCRIPTION

There are described herein methods and systems for spectral identification of a microorganism. The microorganism may be any microscopic living organism that is single-celled, such as but not limited to bacteria, archaea, yeasts, fungi, and molds. A sample of the microorganism is provided on a disposable infrared substrate. The sample contains intact microbial cells having a limited water content level. No drying treatments are applied to the sample, and no reagents are used to reduce or eliminate the original water content of the sample during the sample preparation time. Free water mostly evaporates as soon as the sample is placed on the disposable infrared-compatible substrate, while associated water and bound water remain.

In some embodiments, a vacuum may be applied post-deposition of the microorganism on the disposable infrared substrate for the purpose of removing any remaining free water and associated water in a consistent manner. The infrared spectrum may thus be recorded while the microorganism is under vacuum.

Spectral identification is thus performed based on characteristic spectral fingerprints of intact, whole organisms, with minimal post-culture sample preparation required. Spectral databases of well-characterized strains and multivariate statistical analysis techniques are used to identify unknowns by matching their spectra against those in a reference spectral database.

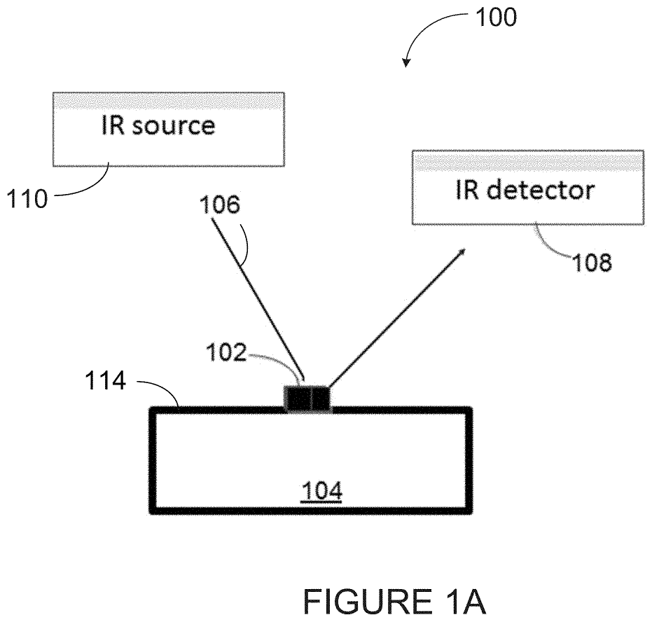

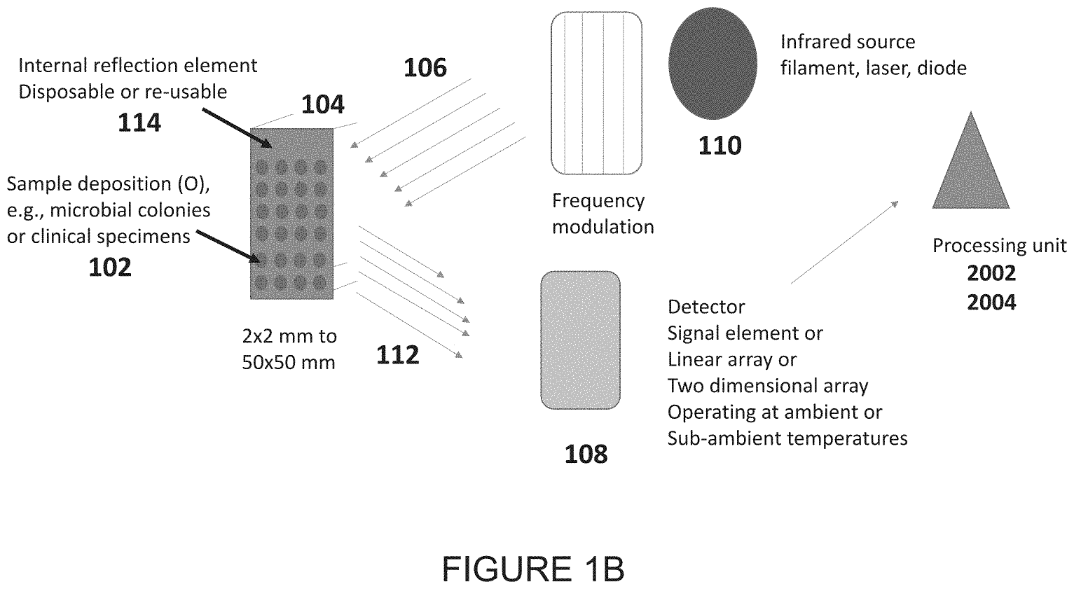

FIG. 1A illustrates an example setup 100 used for spectral identification of a microorganism. The sample 102 sits on a surface 114 of a disposable infrared internal refection substrate 104. The sample 102 may be taken from any known culture medium without breaking the culture medium surface and deposited onto the disposable infrared internal reflection substrate 104 using a transfer device (not shown) such as a sterile toothpick or loop.

The sample 102 may be obtained from a microbial culture, a blood culture, bodily fluids (such as urine and pus, nasal and wound swabs), food, water, air, and the like. The size of the sample 102 should be sufficient to cover a defined area of the disposable infrared internal refection substrate 104. In some embodiments, the sample 102 is sized to be about one tenth ( 1/10) to six millimeters in diameter. Other sample sizes may also be used.

The surface of disposable infrared internal refection substrate 104 is made of a material having an infrared internal reflection property, so that internal reflection of a beam 106, at an angle, through the substrate 104 in contact with the sample 102 returns the internally reflected IR beam toward an infrared detector 108 subsequent to passing through (and being attenuated by) the sample 102. The beam 106 is emitted by an IR source 110. With additional reference to FIG. 1B, in some embodiments the setup 100 is configured for attenuated total reflection infrared (ATR-IR) spectroscopy.

In accordance with an embodiment, the angle of the internal reflection of the beam 106 is greater than a critical angle of incidence above which total internal reflection occurs. Total internal reflection is the phenomenon which occurs when a propagated wave strikes a medium boundary at an angle larger than a particular critical angle with respect to the normal to the surface. If the refractive index is lower on the other side of the boundary and the incident angle is greater than the critical angle, the wave cannot pass through and is entirely reflected. Thus, the critical angle is the angle of incidence above which the total internal reflection occurs.

The disposable infrared internal refection substrate 104 is a substrate composed of a material, such as any one of: germanium, silicon, diamond, zinc selenide, amorphous materials transmitting infrared (AMTIR), thallium bromoidodide (KRS 5), chalcogenides (e.g., chalcogenide glass), halide salts, synthetic diamond film or wafers and any other suitable mid-infrared transmission materials).

A beam 106 of infrared light is propagated through the sample 102 and disposable infrared internal refection substrate 104 generating an evanescent wave perpendicular to the infrared internally propagating through 104. The evanescent wave is attenuated by its interaction with the sample 102. Various optical components, such as lenses and/or mirrors, may be used to direct the beam 106 from a light source 110 to the infrared internal refection substrate 104 and back towards the detector 108 after its propagation through the infrared internal refection substrate 104.

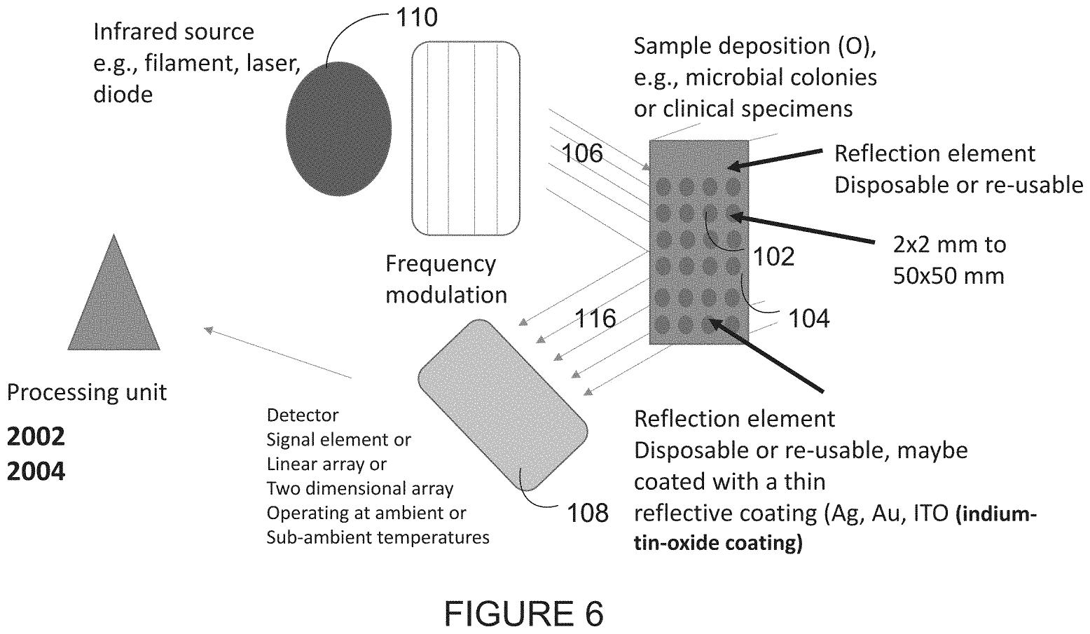

The disposable infrared internal refection substrate 104 may comprise surface infrared reflective properties (e.g., germanium, silicon, chalcogenides) or can be made reflective though deposition of a thin reflective coating (e.g., indium-tin-oxide, gold, aluminum or other materials with infrared reflective properties). The thin coating may be vapor deposited or chemically deposited on the infrared disposable substrate, The thickness of the coating may be on the order of a fraction of the wavelength of the infrared light propagating through the infrared internal refection substrate 104. This allows the disposable infrared internal refection substrate 104 to be also used as a transflection substrate, as illustrated in FIG. 6. In some embodiments the setup 100 is configured for transflection infrared (TFL-IR) spectroscopy as illustrated in FIG. 6.

In some embodiments, at least one thin polymer material is added to infrared internal reflection substrate 104 or the infrared reflective thin coating for attached biomolecules to concentrate the microorganisms near the surface 114 of the internal reflection substrate 104.

In some embodiments, microfluidic devices can be constructed on the disposable infrared substrates to allow simultaneous separation of microorganism from a biological fluid specimen. The microfluidic devices may be single channel or multichannel.

In some embodiments, the disposable infrared internal refection substrate 104 is mounted inside an infrared spectrometer, which may be a Fourier transform infrared (FTIR) spectrometer or a dispersive spectrometer. Any device that can acquire an infrared spectrum in the spectral region between 4000 and 400 wavenumbers and that can be coupled with the spectrometer optical components, such as devices that are filter-based, variable filter array-based, FTIR-based, Fabry-Perot-based and quantum cascade laser (QCL)-based spectrometers, may be used. The light source 110 may be an infrared light source configured to emit infrared light at one or more wavelengths, and the detector 108 may be an infrared detector configured for detecting the reflected beam 112 at a single detection point or a plurality of detection points corresponding to different regions of the sample 102. In some embodiments, the infrared spectrometer is an FTIR spectrometer operating in rapid-scan mode and having an infrared microscope and a focal-plane-array (FPA) detector, such as a 64×64 array of detector elements, referred to herein as an FPA-FTIR spectrometer. In some embodiments, the infrared spectrometer is a Fabry-Perot spectrometer operating and having an infrared array (FPA) detector, such as a 320×256 and 640×480 array of detector elements. In some embodiments, the infrared spectrometer is a dispersive spectrometer that employs a linear variable filter and a pyroelectric detector array.

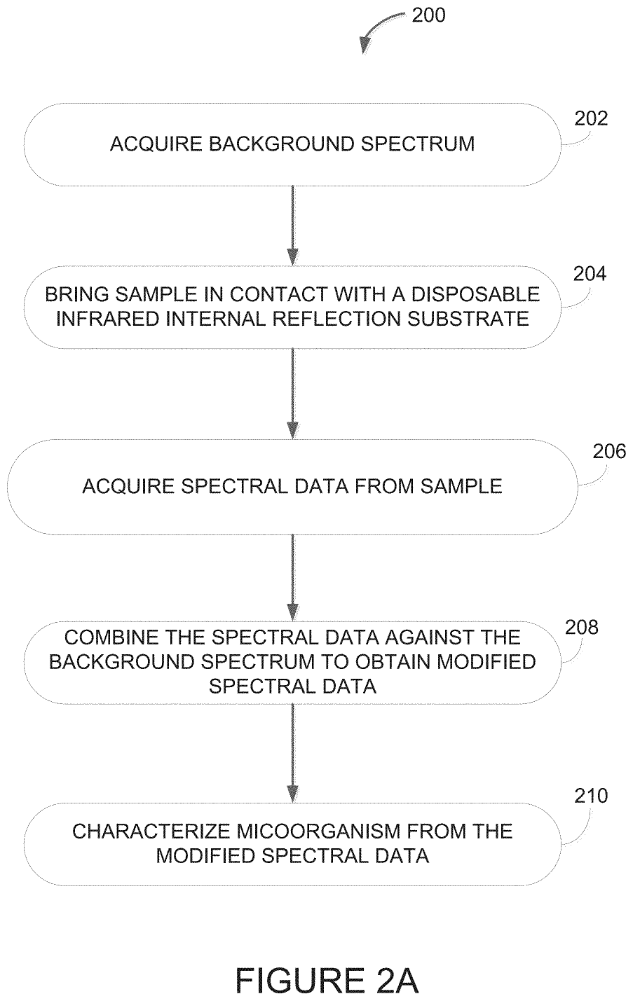

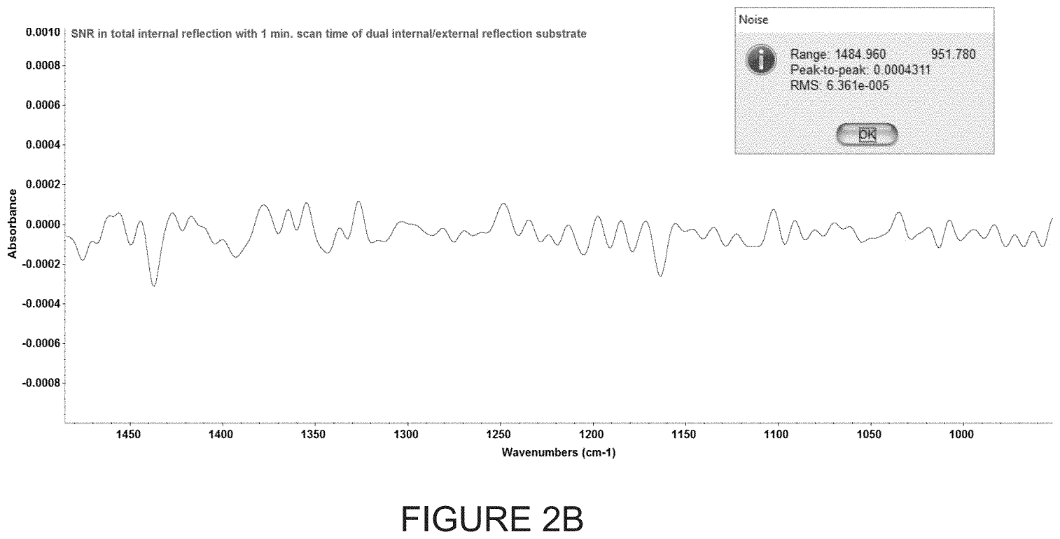

Referring to FIG. 2A, there is illustrated a method 200 for identification of a microorganism using the setup 100. At step 202, a background spectrum is acquired. The background spectrum may measure a water vapor level of the ambient atmosphere in the path between the light source 110 and the detector 108. For example, the beam 106 may be measured by the detector 108 when the surface 114 of the disposable infrared reflective substrate 104 is without the sample. Once the background spectrum has been acquired, as per step 202, the sample 102 is brought into contact with the disposable infrared reflective substrate 104 using any automated and/or manual means, without compromising the integrity of the intact microbial cells, as per step 204. As explained above, the sample 102 may be transferred onto the substrate 104 using any type of transfer device.

At step 206, the spectral data from the sample is acquired no more than a predetermined amount of time after bringing the sample 102 into contact with the infrared internal refection substrate 104 without compromising the integrity of the intact microbial cells. In some embodiments, the predetermined amount of time is less than or equal to one minute. In some embodiments, the predetermined amount of time is selected from a range of about two minutes to about five seconds. In some embodiments, the predetermined amount of time is the minimal time it takes to swab the culture medium, apply the sample to the disposable infrared substrate 104, and press scan on the spectrometer. When automated, the sample 102 may be kept at a very close distance to the infrared internal refection substrate 104 without being in contact there with while the background spectrum is acquired, followed by immediate contact of the sample 102 with the infrared internal refection substrate 104 and acquisition of the spectral data. A full spectral range from 4000 cm⁻¹ to 400 cm⁻¹ may be acquired, even though spectral data from one or more narrower spectral regions may be employed for the purpose of enhancing reproducibility and accuracy of bacterial differentiation. In some embodiments, if it is desired to access spectral regions partially masked by H₂O absorption, for example, the spectral region between 1700 and 1600 cm⁻¹, the H₂O in the sample may be replaced by deuterium oxide (D₂O).

At step 208, the background spectrum and the spectral data are combined to obtain the modified spectral data. Combining the background spectrum and the spectral data may also be viewed as performing a ratio of the spectral data against the background spectrum. The acquisitions are combined to obtain a transmittance spectrum that is then used to produce an absorbance spectrum “A”. The time between the two acquisitions, namely of the background spectrum and the spectral data from the sample, is limited in order to prevent evaporation of the water content from the sample, and to ensure as close a match as possible of the water vapor content of the ambient atmosphere between the two acquisitions. As such, when the background spectrum and the spectral data are combined, water vapor bands are effectively eliminated from the spectral data.

In some embodiments, combining the background spectrum and the spectral data comprises dividing the sample data by the background data (to obtain the transmittance spectrum) and taking a logarithm of the result (to obtain the absorbance spectrum):

A=−log₁₀(sample/background)

The result (“A”) may be viewed as modified spectral data, as the water vapor bands from the sample spectral data have been removed, and it forms the basis of the analysis performed in order to characterize the microorganism, as per step 210.

FIG. 2B is an example of modified spectral data 400 acquired in the absence of a sample. The region 402 shows a peak-to-peak noise level of less than 0.0005 absorbance units. The peak-to-peak noise level is 0.00043 absorbance units for the range of 1406.765 cm⁻¹ to 957.953 cm⁻¹. The root-mean-square (RMS) noise level is 6.4*10⁻⁵.

In some embodiments, step 210 of the method 200 is performed as described in U.S. Pat. No. 9,551,654, the contents of which are incorporated by reference. For example, at least one multi-pixel spectral image of the sample is obtained, wherein each pixel of the image has a corresponding spectrum, and one or more spectra is selected from the spectral image based on one or more spectral characteristics of the corresponding spectrum. The microorganism may be identified by comparing the one or more selected spectra with spectra of reference microorganisms from a database. The modified spectral data is compared to those in the spectral databases containing spectra of pre-characterized isolates. Single or multiple multivariate methods may be employed for the identification of the isolate. Among the multivariate methods are hierarchical cluster analysis (HCA), principal component analysis (PCA), partial least squares (PLS), and spectral search which generate a similarity match between the spectra of unknown isolate and a near identical spectrum in the spectral database. It should be noted that selected spectral regions rather than the full spectrum may be employed in the identification procedure.

The signal-to-noise ratio (SNR) of the spectral data may be improved by performing a greater number of scans of the sample, such as 64, 128, or 256 instead of 4, 16, or 32. However, a greater number of scans means a longer scan time, increasing the difference between the water vapor level in the background spectrum and the spectral data. The method may thus comprise: obtaining an acceptable SNR while minimizing the difference in water vapor level between the background spectrum and the spectral data. In some embodiments, the selected number of scans for the acquisition of the spectral data is 128. Other numbers of scans may also be used. Spectra acquired from lower number of scans can be co-added to improve the SNR.

In some embodiments, the data selected for analysis from the modified spectral data is taken from a range of about 1480 cm⁻¹ to about 800 cm⁻¹. In some embodiments, the range is about 3030 cm⁻¹ to about 2800 cm⁻¹. In some embodiments, the range is about 1770 cm⁻¹ to about 650 cm⁻¹. Other ranges may also be used,

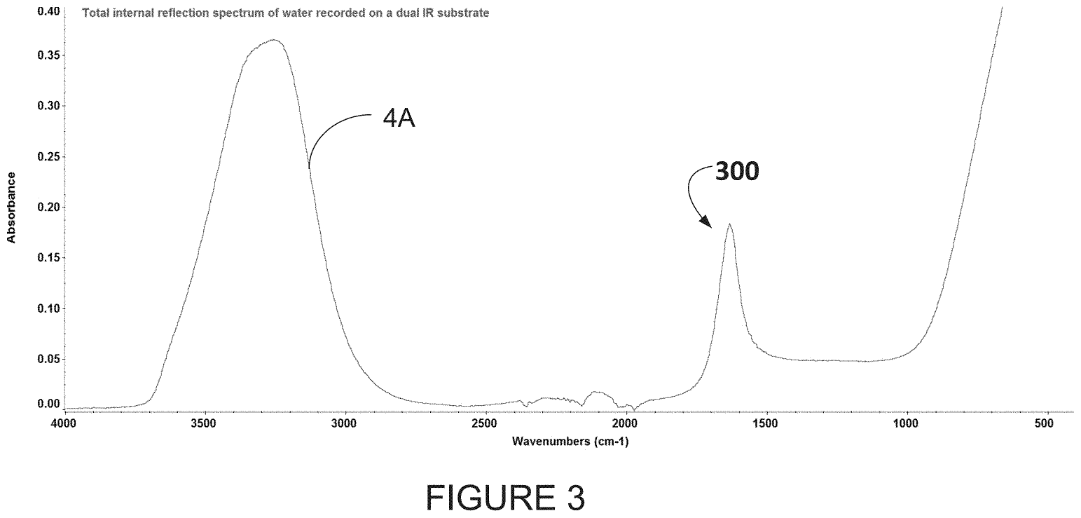

FIG. 3 is an example of a water spectrum 4A acquired by first recording a background spectrum in the absence of a sample and then placing a drop of water on the disposable infrared internal refection substrate 104 and acquiring a second spectrum which is ratioed against the background spectrum and expresses in absorbance values. The signal 300 was acquired by co-adding 64 scans taken during 45 seconds. Note that fewer scans, such as 4, 16, and 32, may be used, and more scans, such as 128 and 256 may be used.

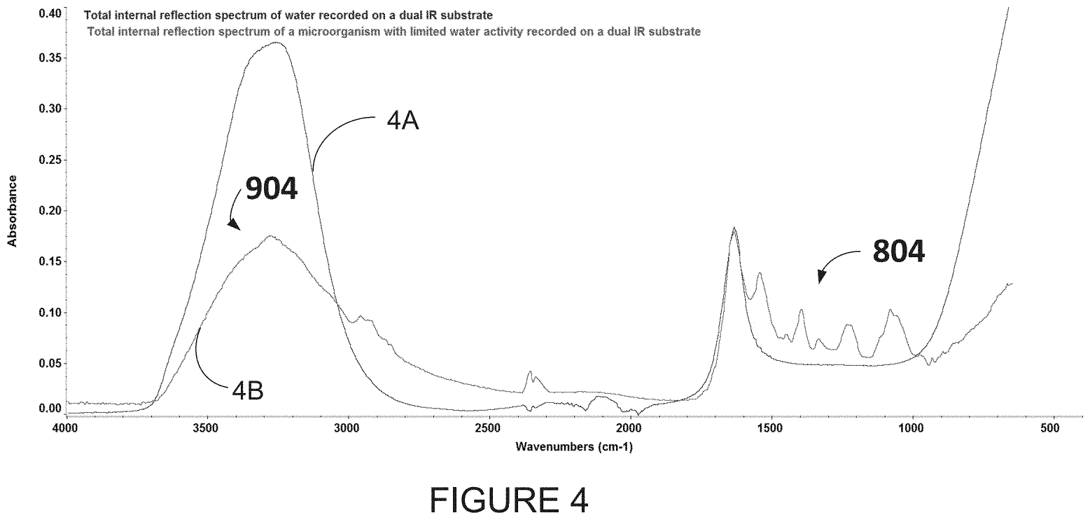

Referring to FIG. 4, the water spectrum 4A of FIG. 3 and a spectrum 4B of a microorganism are each deposited on a disposable ATR-IR substrate using the FTIR spectrometer. Distinct infrared bands of microorganisms in a first region (reference numeral 804) between 980 and 1600 cm⁻¹ and in a second region (reference numeral 904) between 2800 and 3100 cm⁻¹ can be observed. The measurements of region 804 are compared to a second threshold. A measurement for water content of the sample is considered compliant if it is above the second threshold, so as to ensure that the water content of the sample is retained at the time of spectral acquisition. In embodiments in which the threshold is signal intensity in region 804 of 0.4 absorbance units ±0.3 absorbance units. Measurements below the second threshold are indicative of a sample that is too thin (<0.01 absorbance units). The modified spectral data may be rejected as being non-compliant in such a case, Region 904 in FIG. 4 shows an example of the water content of the sample. Validation may be performed visually by comparing the captured signal to another signal or it may be performed automatically by comparing the measured values to the second threshold value.

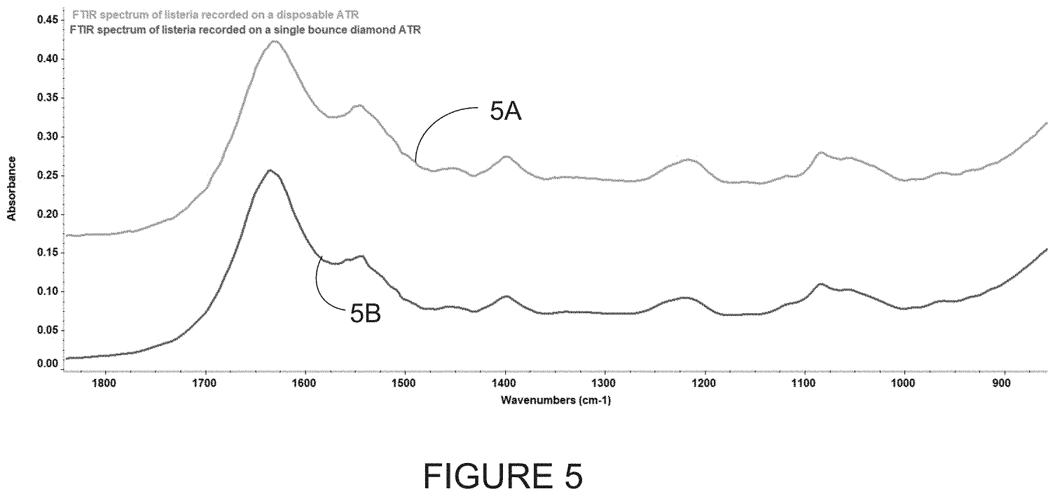

FIG. 5 shows a comparison between a spectra 5A of Listeria grayi recorded on a commercially available single-bounce diamond ATR-FTIR spectrometer equipped with a single element detector and spectra 5B recorded using the disposable infrared internal refection substrate coupled to the same FTIR spectrometer equipped with a single element detector. The spectral quality is comparable and thus may provide the same microbial discriminatory performance as those provided in the PCT Publication No. WO 2017/210783, the contents of which are hereby incorporated by reference.

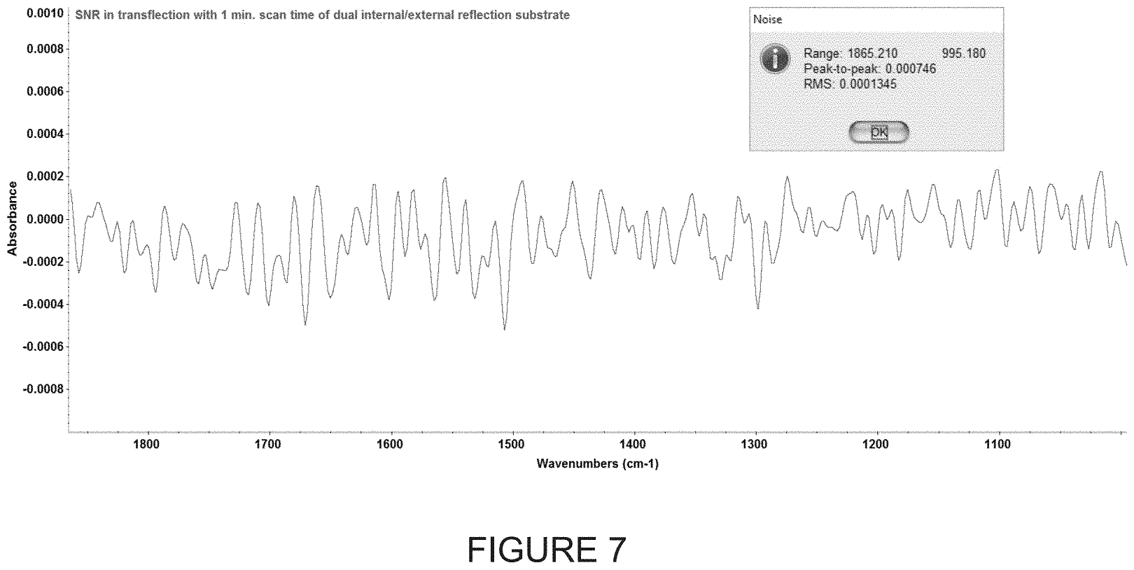

Referring to FIG. 7, a baseline spectrum is shown. The baseline spectrum was recorded using a disposable TFL-IR substrate. The baseline spectrum illustrates the signal-to-noise ratio (SNR) recorded using a portable FTIR spectrometer in a spectral region nominally employed in the differentiation between microorganisms. In this example, the RMS is 0.000134.

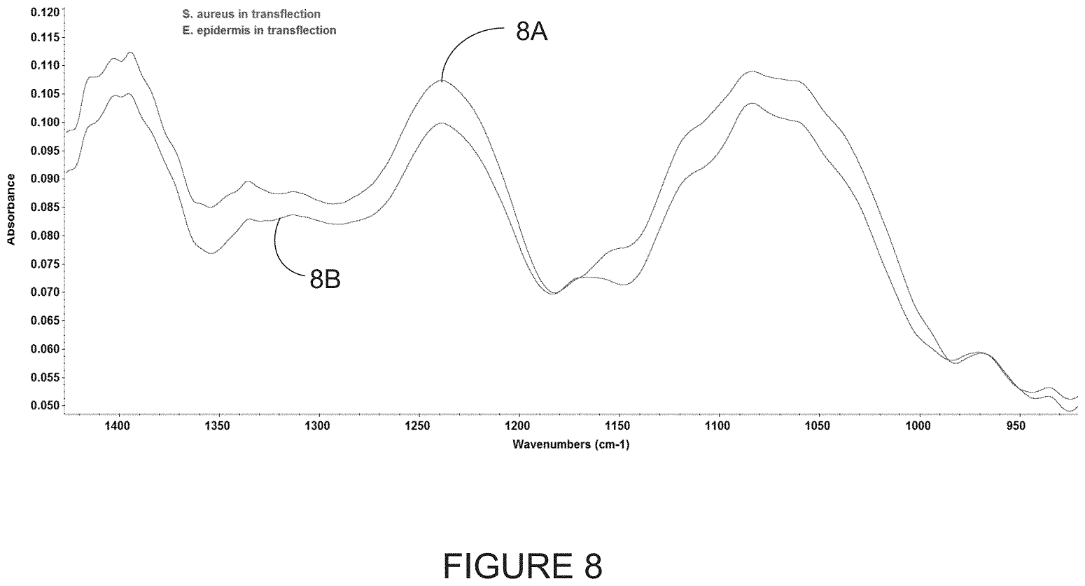

Referring to FIG. 8, a TFL-IR spectra 8A of a S. aureus and a TFL-IR spectra 8B S. epidermis are shown, where the S. aureus and the S. epidermis were deposited on a disposable substrate.

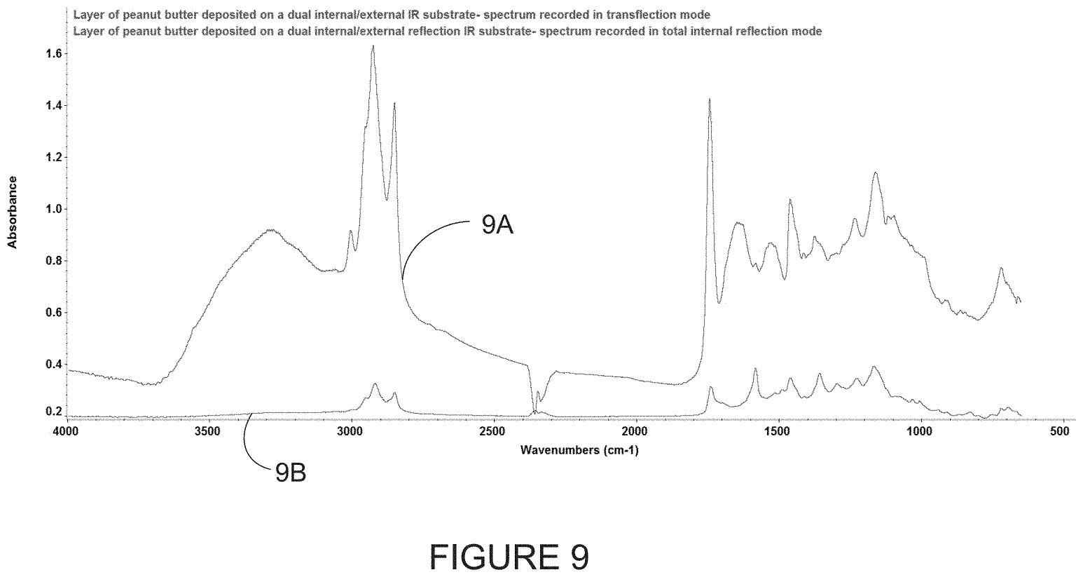

Referring to FIG. 9 a first spectrum 9A is shown for a layer of thick emulsion deposited on a disposable IR substrate, where the first spectrum is recorded in transflection mode. As shown, the absorbance values are high due to the long optical path length. A second spectrum 9B is shown for a layer of the same emulsion, where the second spectrum 9B is recorded by ATR-IR spectroscopy. As shown, the path length of the second spectrum 9B is much shorter than the first spectrum 9A with the transflection measurement.

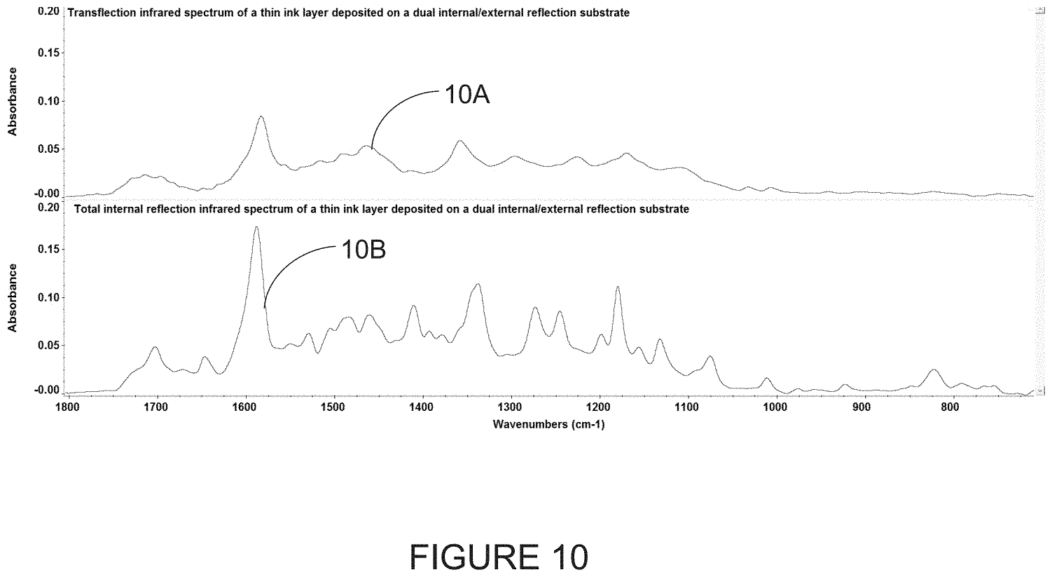

Referring to FIG. 10, a TFL-IR spectrum 10A is shown for a disposable substrate of a thin ink layer. The absorbance values are very low due to the short optical path length. An ATR-IR spectrum 10B of the same thin ink layer is also shown. For the ATR-IR spectrum 10B, the absorbance values are higher than the TFL-IR spectrum 10A due to the acquisition of the spectrum in the thin ink layer in contact with the ATR-IR substrate surface.

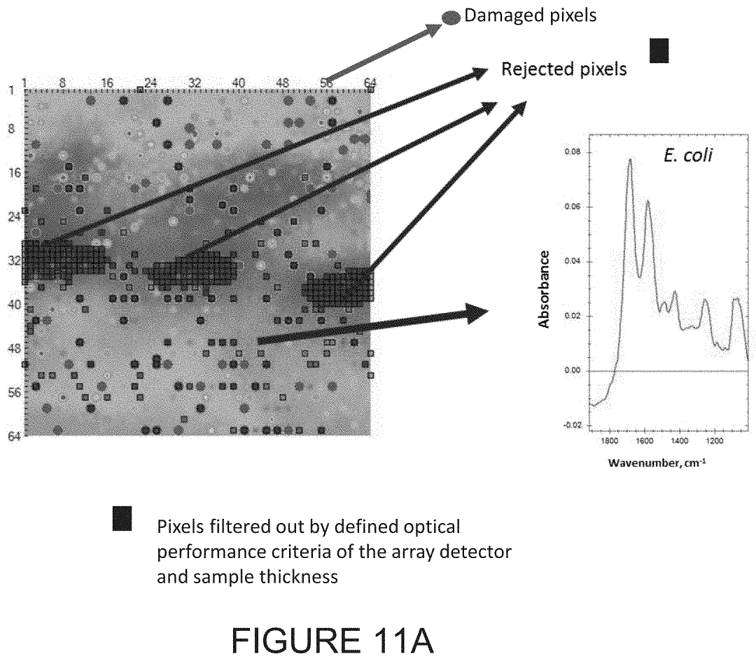

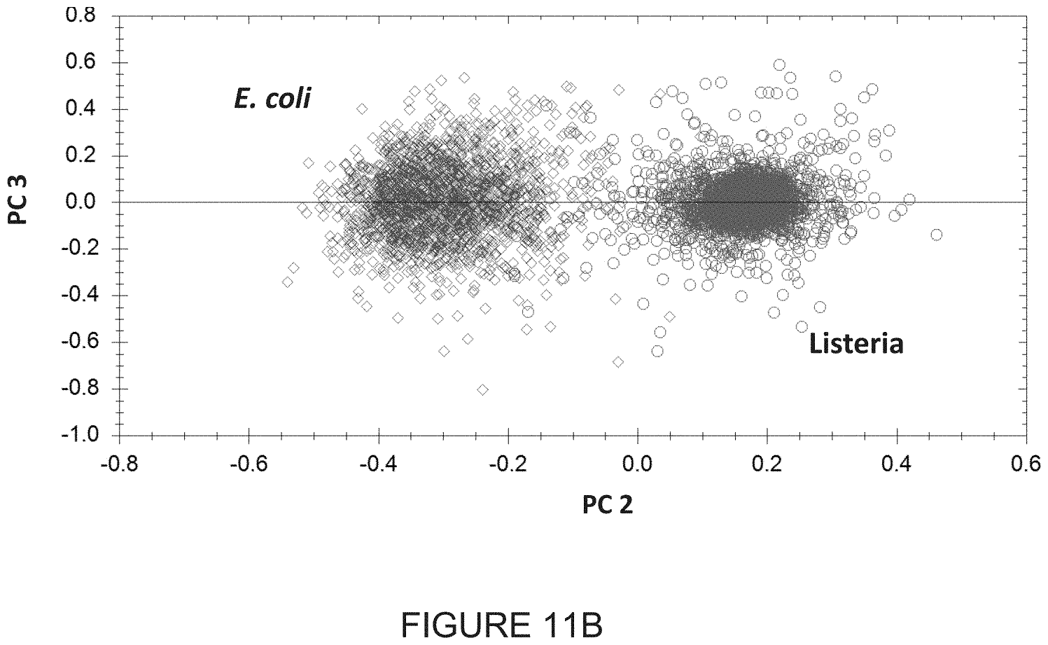

Referring to FIG. 11A a chemical image is shown. The chemical image is generated by plotting the amide I band in the infrared spectral spectra of Listeria grayi recorded using a focal plane array detector (with 64×64 pixels) FTIR spectrometer, The spectral image is recorded from bacteria deposited on a disposable ATR-IR substrate. Arrows show rejected pixels due to damaged pixels and thick sample areas (boxes).

Referring to FIG. 11B, a principal component plot is shown. The principal component plot illustrates the discrimination between E. coli and Listeria grayi based on spectral differences acquired by FPA-FTIR infrared imaging microscopy. The spectra are acquired from microorganisms deposited on a disposable ATR-IR substrate from post pixel filtration.

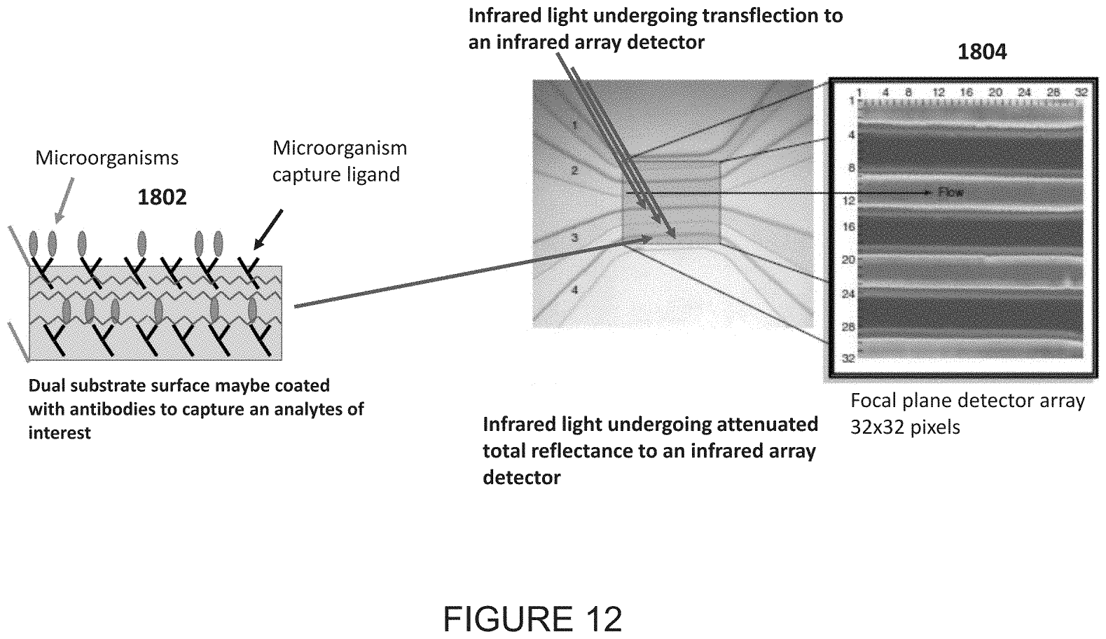

FIG. 12 illustrates an example of the use of a dual purpose disposable transflection infrared substrate integrated into a multi-channel microfluidic device for analysis of biological samples. In this example, the infrared spectra is recorded in a TFL-IR mode to increase pathlength.

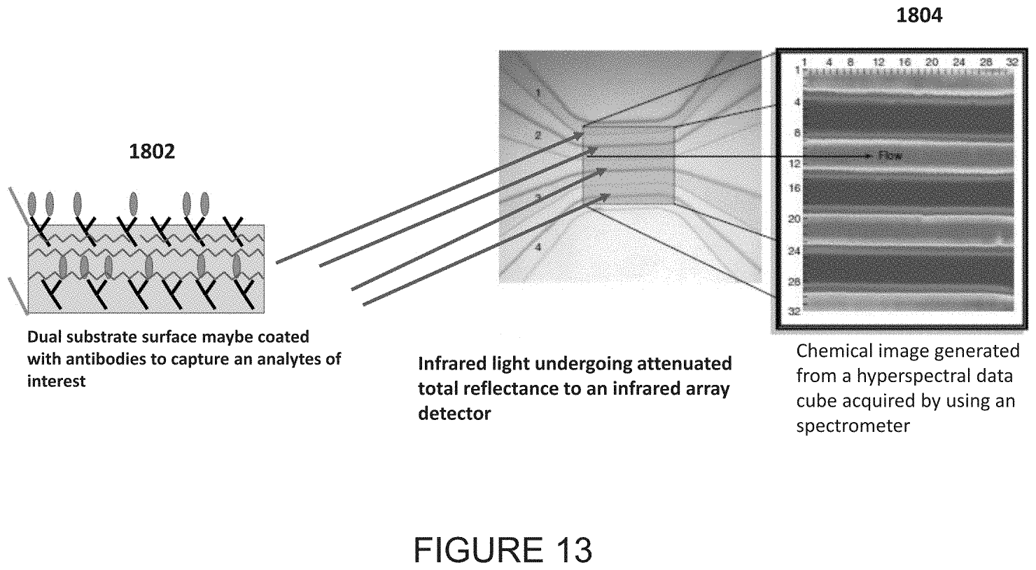

In some embodiments, the disposable infrared internal reflection substrate comprises two surfaces (which may also be referred to as “dual surfaces” or “dual substrate surface”). FIG. 13 illustrates an example of the use of a dual purpose disposable attenuated total reflectance infrared substrate integrated into a multi-channel microfluidic device for analysis of biological samples. In this example, infrared spectra is recorded in an ATR-IR configuration to reduce strong solvent absorption in the microfluidic device. In some embodiments, as shown in FIG. 13, the dual substrate surface is coated with antibodies for the purpose of capturing an analysts of interest.

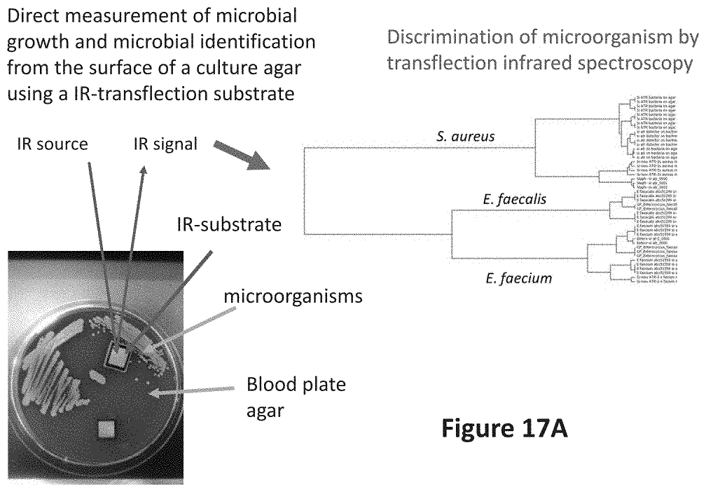

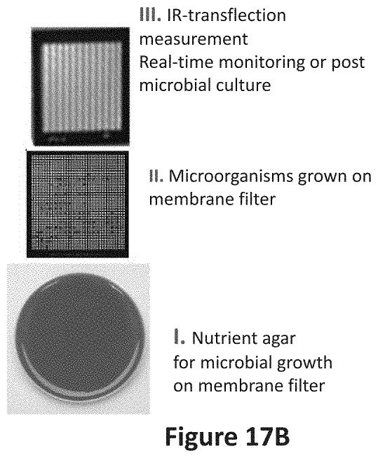

FIG. 17A illustrates an example of use of a dual surface infrared substrate for recording spectra from a enterococci and staphylococci species directly from a nutrient agar. By using dual surface infrared substrate, infrared spectra may be recorded during the growth phase of microorganisms.

FIG. 17B illustrates an example of an infrared measurement for identifying microorganisms from a hydrophobic membrane filter. A plurality of microorganisms may be identified individually, during or subsequent to growth on a hydrophobic membrane filter. The transflection measurements can be recorded using a single-element detector or an array detector, as described elsewhere in this document.

In the creation of a spectral database, the microorganisms may be cultured twice to ensure purity. Isolated colonies with the same morphology may be selected and transferred to the surface of the disposable infrared internal refection substrate for FTIR spectroscopic measurement. The infrared internal refection FTIR spectrum is recorded. Replicate spectra may be obtained and those with the smallest standard deviation from the mean, are added to the database. Additional information may be added to a spectral file header, such as genus, species, strain, antimicrobial profile, growth medium, growth conditions, date, and the like.

In some embodiments, the modified spectral data is compared with spectral data of reference microorganisms obtained using a same culture medium as the sample. The use of another culture medium may result in an altered spectral profile. Therefore, the same media may be used to ensure that the same spectral profile is obtained. Alternatively, spectral data of reference microorganisms may be obtained using a plurality of different culture media, and data from each spectral acquisition are pooled in order to make the reference data culture-media independent.

The method 200 may be used to identify microorganisms from positive blood cultures. While traces of blood in dried samples act as large contaminants, having the blood diluted in water causes the effect to be negligible. FIG. 11B illustrates a principal component (PCA) plot showing differentiation between E. coli (K12) and Listeria grayi based on differences in their infrared internal reflection FTIR spectra.

In some embodiments, the prediction of the identity of an unknown microorganism is carried out by infrared internal refection-FTIR spectral analysis independent to the MALDI-TOF MS analysis. The identification of the unknown microorganism by the two independent means can further enhance the reliability of the identification by MALDI-TOF MS.

In some embodiments, other spectral data is acquired from another spectroscopic technique- such as ¹H (proton), ¹³C, ³¹P or ¹⁵N nuclear magnetic resonance (NMR) spectroscopy, including solid-state high-resolution magic angle spinning (HRMAS) NMR. The infrared internal refection-FTIR data may thus be used to identify the spectral features responsible for the differentiation between two types of microorganisms. Subsequently, or in tandem, other spectral data from other spectroscopic techniques can be utilized to identify the biomarker(s) associated with the infrared spectral features. In some embodiments, spectra generated from stitching of multiple spectral data sets from the above-mentioned techniques can be subjected to analysis with the use of a FSA after spectral pre-processing, including normalization. Individually or combined, these pre-processing methods increase the reliability of microbial identification by multispectral domain spectroscopy.

It should be noted that the sample may have been previously treated using various processes, such as those associated with clinical samples, subcultures, and/or frozen samples. For example, immuno-capture methods for extraction of microorganism from blood (or other bodily fluids) employing magnetic beads form a bacteria-bead complex can be directly measured by internal reflection FTIR spectroscopy.

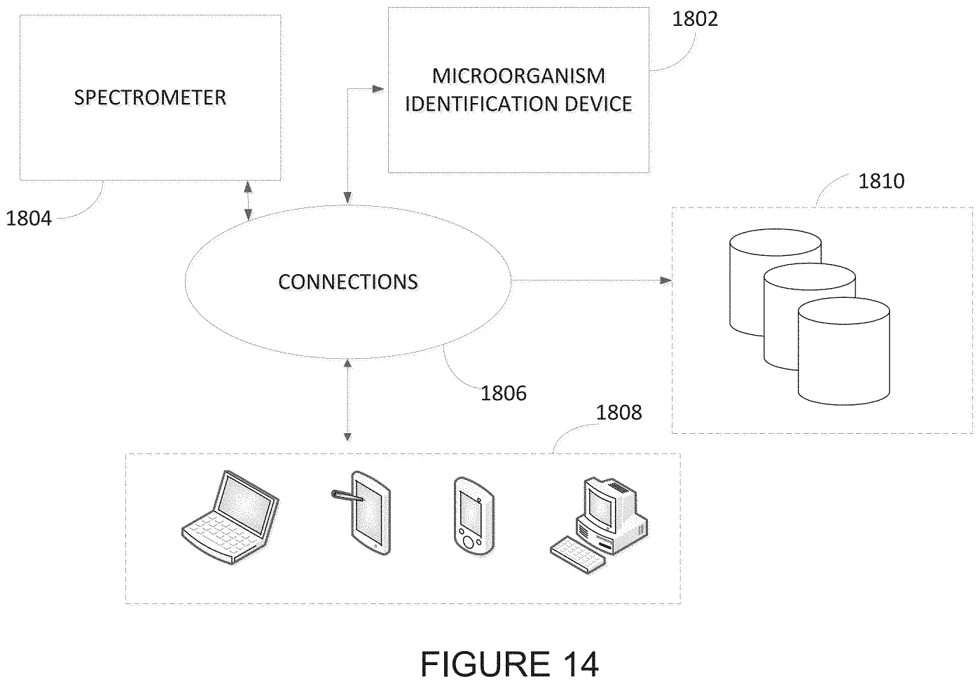

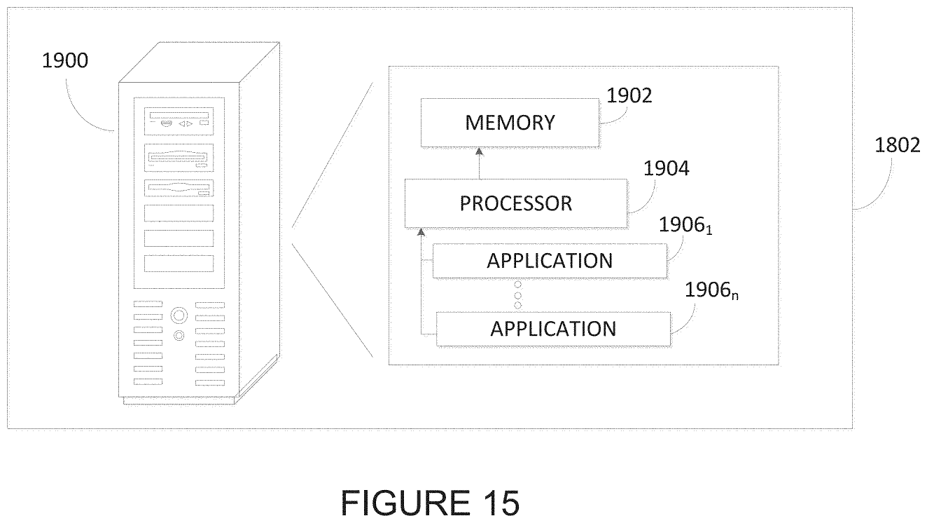

Referring FIGS. 14 and 15, a system for spectral identification of microorganisms will now be described. In FIG. 14, there is illustrated a microorganism identification device 1802 operatively connected to spectrometer 1804. The microorganism separation device 1802 may be provided separately from or incorporated within the spectrometer 1804. For example, the microorganism separation device 1802 may be a microfluidic device capable of separating the microorganisms from a biological fluid. The device may be integrated with the spectrometer 1804. The spectrometer 1804 may be any instrument capable of acquiring infrared spectral data from an object, such as but not limited to an FTIR spectrometer, Some example spectral acquisition parameters are as follows:
Resolution: 8 cm⁻¹
Zero filling: 0-8 orders
Detector type: DTGS or MCT or FPA (operating at ambient or sub-ambient temperatures)
Detector gain: 1-4
Apodization: triangular or Happ-Ganzel
Number of scans: 8-256
Time of acquisition: 10-300 seconds
Background (before each sample: 4-128 scans)
SNR: >1,000:1 (or 1 mAu between 1380 and 980 cm⁻1) (100% line, 64 scans/8 cm⁻¹) with residual water vapor<0.005 Au

In some embodiments, the following protocol may be used for acquiring the background spectrum and spectral data with the spectrometer 1804:
1. Turning on the instrument and letting it warm up.
2. Launching the software on the computer and setting the spectral acquisition parameters to:

Number of scans: 64 scans (or another value, as desired)

Resolution: 4-8 cm⁻¹.
3. Collecting a background spectrum (noting that the surface of the disposable infrared internal reflection substrate must be bare, clean & dry).
4. Collecting a small amount of bacteria (˜1-5 colonies) from a culture plate using a sterilized toothpick or loop without breaking the culture medium surface.
5. Spreading the collected bacteria on the surface of the disposable infrared internal reflection substrate (˜2-8 mm in diameter).
6. Pressing “Scan sample” to collect the spectral data.
7. Discarding or cleaning the disposable infrared reflective surface by wetting the bacteria with a disinfecting fluid (70% ethanol or bleach),
8. Wiping the bacteria off using a Kimwipe.
9. Repeating steps 3 through 8 for each subsequent sample and acquiring a spectrum of a preselected reference strain after every 30 samples. These numbers are purely illustrative and may be varied.
10. Cleaning the surface of the disposable infrared substrate by the procedure in step 8 (or discarding the infrared substrate) and turning off the instrument,

The following experimental protocol was used for infrared internal reflection FTIR spectral acquisition. Gram-positive isolates were sub-cultured on 5% sheep's blood agar for 18-24 h at 35° C. With certain exceptions, Gram-negative isolates were sub-cultured on 5% sheep's blood agar or MacConkey agar for 18-24 h at 35° C. Following incubation, 1-5 isolated colonies were collected from the agar surface and spread on the surface of the disposable infrared internal reflection substrate and placed in the FTIR spectrometer and a spectrum was immediately recorded using a spectral acquisition time of 45 seconds. For each culture plate, 2-3 replicate spectra were acquired from different colonies.

Referring back to FIG. 14, various types of connections may be provided to allow the microorganism identification device to communicate with the spectrometer. For example, the connections may comprise wire-based technology, such as electrical wires or cables, and/or optical fibers. The connections may also be wireless, such as RF, infrared, Wi-Fi, Bluetooth, and others. Connections may therefore comprise a network, such as the Internet, the Public Switch Telephone Network (PSTN), a cellular network, or others known to those skilled in the art. Communication over the network may occur using any known communication protocols that enable devices within a computer network to exchange information. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol), and Ethernet. The connections 1806 may also use various encryption means to protect any of the data acquired and/or transferred.

The microorganism identification device may be accessible remotely from any one of a plurality of devices over connections. The devices may comprise any device, such as a personal computer, a tablet, a smart phone, or the like, which is configured to communicate over the connections. In some embodiments, the microorganism identification device 1802 may itself be provided directly on one of the devices, either as a downloaded software application, a firmware application, or a combination thereof.

One or more databases may be integrated directly into the microorganism identification device or any one of the devices, or may be provided separately therefrom (as illustrated). In the case of a remote access to the databases, access may occur via connections taking the form of any type of network, as indicated above. The various databases described herein may be provided as collections of data or information organized for rapid search and retrieval by a computer. The databases may be structured to facilitate storage, retrieval, modification, and deletion of data in conjunction with various data-processing operations. The databases may be any organization of data on a data storage medium, such as one or more servers or long-term data storage devices. The databases illustratively have stored therein spectral data for reference microorganisms used for comparison with spectral data of unknown samples.

The microorganism identification device illustratively comprises one or more servers. For example, a series of servers corresponding to a web server, an application server, and a database server may be used. These servers are all represented by server. The server may be accessed by a user, such as a technician or laboratory worker, using one of the devices, or directly on the system via a graphical user interface. The server may comprise, amongst other things, a plurality of applications running on a processor coupled to a memory. It should be understood that while the applications presented herein are illustrated and described as separate entities, they may be combined or separated in a variety of ways.

The memory accessible by the processor may receive and store data. The memory may be a main memory, such as a high-speed Random Access Memory (RAM), or an auxiliary storage unit, such as a hard disk, a floppy disk, or a magnetic tape drive. The memory may be any other type of memory, such as a Read-Only Memory (ROM), or optical storage media such as a videodisc and a compact disc. The processor may access the memory to retrieve data. The processor may be any device that can perform operations on data. Examples are a central processing unit (CPU), a front-end processor, a microprocessor, and a network processor. The applications are coupled to the processor and configured to perform various tasks. An output may be transmitted to the devices.

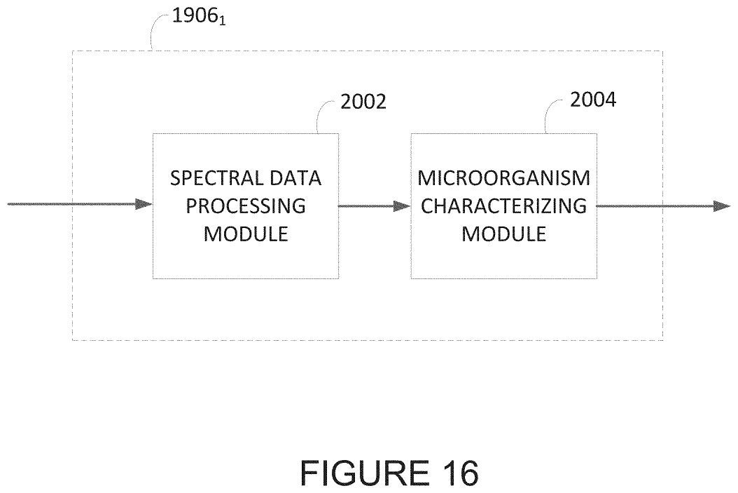

FIG. 16 is an exemplary embodiment of an application running on the processor. The application illustratively comprises a spectral data processing module 2002 and a microorganism characterizing module 2004. The spectral data processing module 2002 is configured for receiving the background spectrum and the spectral data. The spectral data processing module 2002 may also be configured for combining the background spectrum and the spectral data to produce the modified spectral data. In some embodiments, the spectral data processing module is further configured for validating the modified spectral data, for example by comparing water vapor level, sample water content, and/or sample biomass to a threshold or a reference value. Some of the mathematical operations performed by the spectral data processing module 2002 on the background spectrum and/or spectral data include, but are not limited to, first derivatives, vector normalizations (4000-400 cm⁻¹), and cubic interpolation (with data spacing of 0.1-32).

The microorganism characterizing module 2004 may be configured to receive the modified spectral data and to perform microorganism characterization by comparing the modified spectral data to reference spectral data of known microorganisms. In some embodiments, the microorganism characterizing module 2004 is configured to use target spectral regions in the modified spectral data pre-selected by applying a feature selection algorithm to training data as per U.S. Pat. No. 9,551,654. For example, an FSA is employed to identify the significant biochemical markers that are more relevant than the proteins in microbial identification. The comprehensive information content in the FTIR spectra can differentiate between types of bacteria at different levels of classification (genus, species, strain, serotype, and antimicrobial resistance characteristics and in some cases genotypic characteristics). Based on the FSA, spectral regions attributed to specific class of biomolecules (example, polysaccharides, lipids, proteins or nucleic acids) may then be identified to increase the resolution power of MALDI-TOF MS in its ability to differentiate between closely related genera, such as E. coli and Shigella.

In some embodiments, a grid-greedy feature selection algorithm is used with three regions of a minimum size of 20 wavenumbers (6 features) and a maximum size of 92 wavenumbers (24 features) per region. All possible combinations of such regions are evaluated between 3050 and 2700 cm⁻¹ and between 1780 and 400 cm⁻¹ and the region with the highest LOOCV-KNN classification score is selected. The greedy portion of the algorithm examines combinations of adjacent features following the path of greatest improvement. The forward selection begins by evaluating the single feature with the highest classification score, followed by adding features one at a time which keeps the score at a maximum. The routine stops when the classification score is no longer improved by adding features. The search may continue for a minimum of 6 features (1% of the total number of features) even if there is no further improvement in classification score in order to minimize over-fitting of the training data. Other feature selection algorithms may also be used.

The methods and systems described herein employ a simple and universally applicable protocol that requires minimal sample preparation and no reagent beyond a culturing step. The methods may be used with a high degree of automation and is amenable to micro colony analysis. They may produce a fast turnaround time at a low cost per test, and are capable of detecting biochemical differences between antibiotic-resistant and susceptible bacterial strains in the absence or in the presence of the antibiotic.

The methods and systems described herein may also be used for the identification of clinical isolates from positive blood cultures. Indeed, as long as there is sufficient microorganism biomass that can be obtained from a positive blood culture, direct identification of bacteria may be performed using reflection-FTIR spectroscopy as described herein.

In some embodiments, the FTIR spectroscopic methods using a disposable infrared substrate and systems described herein can be complemented by MALDI-TOF MS and/or HRMAS NMR (high-resolution magic-angle spinning NMR), for example, for the discrimination between MRSA and MSSA, VRE and VSE, and E. coli and Shigella spp. The methods and systems may also be used for the identification of Shiga-toxin-producing E. coli (STEC).

In some embodiments, the disposable infrared internal reflection substrate can be used in conjunction with a portable FTIR spectrometers to perform the methods and implement the systems described herein.

In some embodiments, the disposable infrared internal reflection substrate may be used to record FTIR spectra by transflection spectroscopy in conjunction with a portable FTIR spectrometers to perform the methods and implement the systems described herein.

The disposable internal reflection substrate may be used in conjunction with a portable infrared spectrometer equipped with an array detector operating at ambient or sub ambient temperatures. Spectra recorded from bacteria deposited on the infrared internal reflection disposable substrates can compensate for the limitations of MALDI-TOF MS, such as the inability to discriminate between E. coli and Shigella. It should be appreciated that microbiology laboratories may effectively employ their current MALDI-TOF MS SOP with the methods and systems described herein to overcome MALDI-TOF MS limitations. In particular, MALDI-TOF MS is generally unable to discriminate between antibiotic-sensitive and antibiotic-resistant bacteria. The techniques and methods described herein may be used to discriminate between antibiotic-sensitive and antibiotic-resistant bacteria.

The above description is meant to be exemplary only, and one skilled in the relevant arts will recognize that changes may be made to the embodiments described without departing from the scope of the invention disclosed. For example, the blocks and/or operations in the flowcharts and drawings described herein are for purposes of example only. There may be many variations to these blocks and/or operations without departing from the teachings of the present disclosure. For instance, the blocks may be performed in a differing order, or blocks may be added, deleted, or modified. While illustrated in the block diagrams as groups of discrete components communicating with each other via distinct data signal connections, it will be understood by those skilled in the art that the present embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present embodiment. The present disclosure may be embodied in other specific forms without departing from the subject matter of the claims. Also, one skilled in the relevant arts will appreciate that while the systems, methods and computer readable mediums disclosed and shown herein may comprise a specific number of elements/components, the systems, methods and computer readable mediums may be modified to include additional or fewer of such elements/components. The present disclosure is also intended to cover and embrace all suitable changes in technology. Modifications which fall within the scope of the present invention will be apparent to those skilled in the art, in light of a review of this disclosure, and such modifications are intended to fall within the appended claims.

1. A method for spectral identification of a microorganism, the method comprising:
acquiring a background spectrum to measure a water vapor level of an ambient atmosphere in the absence of a sample;
bringing the sample containing the microorganism into contact with a disposable infrared internal reflection substrate, the sample having intact microbial cells;
acquiring spectral data from the sample using internal reflection infrared spectroscopy no more than a predetermined time after having acquired the background spectrum;
combining the background spectrum and the spectral data, thereby producing modified spectral data; and
characterizing the microorganism using the modified spectral data.

2. The method of claim 1, wherein the disposable infrared internal reflection substrate is composed of any one of germanium, silicon, amorphous materials transmitting infrared, chalcogenides, zinc selenide and halide salts.

3. The method of claim 2, wherein the disposable infrared internal reflection substrate is coated with an infrared reflective thin coating.

4. The method of claim 3, wherein the infrared reflective thin coating is vapor deposited or chemically deposited.

5. The method of claim 4, wherein the infrared reflective thin coating is indium-tin-oxide or gold.

6. The method of claim 5, wherein at least one thin polymer material is added to the infrared internal reflection substrate for attached biomolecules to concentrate the microorganisms near a surface of the internal reflection substrate.

7. The method of claim 1, wherein the spectral data is acquired from the sample prior to or after having added a MALDI-TOF MS chemical matrix thereto.

8. The method of claim 1, wherein the sample has a limited free water content and an intact associated and bound water content.

9. The method of claim 1 wherein the microorganism of the sample has a water activity of less than 0.999 percent.

10. The method of claim 1, further comprising applying a vacuum to the sample on the disposable infrared internal reflection substrate prior to acquiring the spectral data from the sample.

11. The method of claim 1, further comprising applying a vacuum to the sample on the disposable infrared internal reflection substrate coated with a thin layer of infrared reflective coating and prior to acquiring the spectral data from the sample.

12. The method of claim 1, wherein the disposable infrared internal reflection substrate comprises one or more microfluidic devices for allowing simultaneous separation of the microorganism from the sample.

13. A method for spectral identification of a microorganism, the method comprising:
acquiring a background spectrum to measure a water vapor level of an ambient atmosphere in the absence of a sample;
bringing the sample containing the microorganism into contact with a disposable infrared internal reflection substrate coated with an infrared reflective coating, the sample having intact microbial cells;
acquiring spectral data from the sample using transflection infrared spectroscopy no more than a predetermined time after having acquired the background spectrum;
combining the background spectrum and the spectral data, thereby producing modified spectral data; and
characterizing the microorganism using the modified spectral data.

14. The method of claim 13, wherein the disposable infrared internal reflection substrate is composed of any one of germanium, silicon, amorphous materials transmitting infrared, chalcogenides, zinc selenide and halide salts.

15. The method of claim 14, wherein the infrared reflective coating is vapor deposited or chemically deposited.

16. The method of claim 15, wherein the infrared reflective coating is indium-tin-oxide or gold.

17. The method of claim 16, wherein at least one thin polymer material is added to the infrared internal reflection substrate for attached biomolecules to concentrate the microorganisms near a surface of the internal reflection substrate.

18. The method of claim 13, wherein the spectral data is acquired from the sample prior to or after having added a MALDI-TOF MS chemical matrix thereto.

19. The method of claim 13, wherein the sample has a limited free water content and an intact associated and bound water content.

20. The method of claim 13, wherein the microorganism of the sample has a water activity of less than 0.999 percent.

21. The method of claim 13, further comprising applying a vacuum to the sample on the disposable infrared internal reflection substrate prior to acquiring the spectral data from the sample.

22. The method of claim 13, further comprising applying a vacuum to the sample on the disposable infrared internal reflection substrate coated with a thin layer of infrared reflective coating and prior to acquiring the spectral data from the sample.

23. The method of claim 13, wherein the disposable infrared internal reflection substrate comprises one or more microfluidic devices for allowing simultaneous separation of the microorganism from the sample.

24. A system for spectral identification of a microorganism, the system comprising a processing unit; and
a non-transitory computer-readable memory having stored thereon program instructions, the program instructions are executable by the processing unit for:
acquiring a background spectrum to measure a water vapor level of an ambient atmosphere in the absence of a sample,
acquiring spectral data from the sample, using at least one of internal reflection infrared spectroscopy or transflection infrared spectroscopy, no more than a predetermined time after having acquired the background spectrum, the sample having been brought into contact with a disposable infrared reflective substrate and having intact microbial cells,
combining the background spectrum and the spectral data, thereby producing modified spectral data, and
characterizing the microorganism using the modified spectral data.
